(19) United States
(12) Patent Application Publication
(10) Pub. No.: US 2026/0158760 A1
(43) Pub. Date: Jun. 11, 2026

(54) Roll Assembly for Finishing a Windable, In Particular Pre-Bonded, Sheet Material, and Corresponding Sheet Material

(71) Applicants: Matthews International GmbH, Vreden (DE); Matthews International Corporation, Pittsburgh, PA (US)

(72) Inventors: Klaus-Dieter Kurt THIEL, Viersen (DE); Achim WANDKE, Kempen (DE); Achim VON WIRTH, Erkelenz (DE)

(21) Appl. No.: 18/721,980

(22) PCT Filed: Dec. 12, 2022
(86) PCT No.: PCT/DE2022/100942

§ 371 Date: Jun. 20, 2024

(30) Foreign Application Priority Data
Dec. 23, 2021 (DE) 10 2021 134 589.8

(51) Int. Cl. B31F 1/07; A61F 13/511

(52) U.S. Cl. CPC B31F 1/07; A61F 13/51104; B31F 2201/0733; B31F 2201/0738; B31F 2201/0743

(57) ABSTRACT

The invention relates to a roll assembly for finishing a windable, in particular pre-bonded, sheet material, comprising a first and a second embossing roll, which form a roll nip therebetween for embossing the sheet material; wherein the first roll comprises a plurality of projections for producing structural elements in the sheet material and the second roll comprises a plurality of indentations for receiving the projections; wherein a roll nip portion formed between the first and the second embossing roll is designed to produce a border of locally bonded sheet material which surrounds the structural element at least in part; wherein the roll nip portion comprises at least one first conical bonding portion and at least one bonding portion formed substantially in parallel with the roll axes.

The present invention relates to a roll assembly for finishing a windable, in particular pre-bonded, sheet material, comprising a first and a second embossing roll, which form a roll nip therebetween for embossing the sheet material; wherein the first roll comprises a plurality of projections for producing structural elements in the sheet material and the second roll comprises a plurality of indentations for receiving the projections; wherein a roll nip portion formed between the needle and the indentation is designed to produce a border of locally bonded sheet material which surrounds the structural element at least in portions; wherein the roll nip portion comprises at least one first conical bonding portion. The invention further relates to a corresponding sheet material.

From US 2018/0 228 666 A1 , a device for producing a liquid-absorbing nonwoven fabric with three-dimensional structures and openings is already known. The device described therein comprises a plurality of conical needles which are engaged with complementary conical indentations. In the conical nip thus formed, the material to be embossed is compressed on the one hand, and on the other hand the tip of the needle creates openings in the nonwoven fabric.

However, the disclosed device has the disadvantage that the material to be embossed is only inadequately clamped when the openings are created, so that the material can slip in the nip during the creation of the openings and thus only a low level of uniformity of the structures in the embossed material can be achieved. Furthermore, the structures created in the nonwoven fabric only have a uniform compression intensity, so that when configuring the nonwoven fabric with regard to absorbency and stability, only a compromise solution can be considered.

It is therefore the object of the invention to improve a roll assembly or a windable sheet material in such a way that either has improved product properties and can be produced more economically.

The object is achieved by the features of the independent claims. Advantageous embodiments of the invention are the subject matter of the drawings, the description and the dependent claims.

Accordingly, it is provided that the roll nip portion has at least one bonding portion formed substantially parallel to the roll axes. The invention has the advantage that the bonding portion formed parallel to the roll axes enables a better alignment of the rolls to one another in the production process. The portion parallel to the roll axes fixes the rolls to each other and thus ensures a self-centering effect. As a result, the rolls can be operated at higher speeds than the device known from the prior art. At the same time, the bonding portion, which is designed parallel to the roll axes, ensures improved material clamping so that the material cannot slip over the conical regions and the openings can therefore be produced with greater precision. Furthermore, the conical bonding portion on the one hand and the bonding portion formed parallel to the roll axes on the other hand allow regions of different compression to be created in the material, so that on the one hand a high material stability and on the other hand a pleasant feel or good absorption properties can be realized. The sheet material can, for example, be a nonwoven fabric. The nonwoven fabric can be hydrophilic or hydrophobic. The sheet material can also be tissue or film. The sheet material can be single-layered, multi-layered and/or laminated.

It can be provided that the roll nip portion has a stepped profile. The stepped profile can have a plurality of conical and a plurality of bonding portions formed substantially parallel to the roll axes.

It can be provided that the conical bonding portion encloses the structural element in a ring shape. It is also possible that the bonding portion, which is aligned substantially parallel to the roll axes, surrounds the conical bonding portion at least in portions. The bonding portion, which is aligned substantially parallel to the roll axes, can be located on the plane of the pitch circle diameter of the first and second embossing rolls. The bonding portion, which is aligned substantially parallel to the roll axes, can extend over the entire roll width and can be provided wherever none of the rolls has a projection or a indentation.

It is conceivable that the projections are designed as needles. The needles can be designed to create openings. If the projections are designed as needles, the structural elements are consequently designed as openings. The material clamping by the conical and the bonding portion, which is essentially parallel to the roll axes, enables the needles to create the openings in the sheet material in a precise and accurate manner. Alternatively, the projections can be designed as embossing elevations, which are intended to produce structural elements designed as elevations. If the projections are designed as embossing elevations, the structural elements are consequently designed as elevations. In this case too, the good material clamping allows the elevations to be introduced into the material very precisely.

It is conceivable that the first conical bonding portion is formed between a conical proximal flank portion of the projections and a conical mouth portion of the indentation. The projection may be located on a base region. The projection may extend radially away from the roll surface starting from the base region. The base region may have a greater width than the largest width of the projection. The proximal flank portion of the projections may be provided in a foot region of the projections adjacent to the base region. The indentation may have a substantially cylindrical portion which extends radially into the roll surface. In a mouth region to the roll surface, the indentation can have a bevel on its upper side, which forms the conical opening portion of the indentation. The projection may have a round cross section. Alternatively, the projection may have an oval cross section. The projection can thus be essentially conical.

It can be provided that the bonding portion formed substantially parallel to the roll axes is formed between a saddle region of the projections formed substantially parallel to the roll axes and a shoulder region of the indentation formed substantially parallel to the roll axes. The saddle region can be formed by an upper side of the embossing base. The saddle region may extend from the foot of the projections to an outer edge of the base region. The outer edge of the base region may have a bevel. The shoulder region may extend horizontally, i.e. parallel to the roll axes, from the upper edge of the conical mouth portion. The shoulder region and the saddle region can be aligned parallel to each other.

It is conceivable that the conical bonding portion directly adjoins the bonding portion which is formed substantially parallel to the roll axes. Thus, the roll nip portion creating the border can be arranged in a transition region between the foot region of the projections and the base region.

It is conceivable that the roll nip portion further comprises a second conical bonding portion which, opposite the first conical portion, adjoins the bonding portion formed parallel to the roll axes, wherein the first and the second conical bonding portion extend in opposite directions away from the bonding portion formed parallel to the roll axes. The second conical bonding portion can be formed by the bevel provided on the outer edge of the embossing base and a conical embossing elevation portion adjacent to the bevel.

Furthermore, it can be provided that the second roll has a plurality of embossing elevations for producing elevations in the sheet material and the first roll has a plurality of recesses for receiving the embossing elevations. The second roll may have a plurality of first embossing projections having a first cross section and a plurality of second embossing projections having a second cross section, wherein the second cross section differs from the first cross section. The first cross section can have an oval basic shape. The second cross section can have a round basic shape. The embossing elevations may have angled side walls and a flat top. A radius of 0.3 mm can be provided between the side walls and the flat top.

Furthermore, it can be provided that the second conical bonding portion is formed between a conical proximal flank portion of the embossing elevation and a conical mouth portion of the recess.

It is conceivable that a roll nip thickness in the bonding portion formed substantially parallel to the roll axes is between 0.01 mm and 0.03 mm, preferably 0.02 mm. It can be provided that the roll nip thickness in the bonding portion formed substantially parallel to the roll axes is smaller than the roll nip thickness in the first and/or second conical bonding portion. A smaller roll nip thickness in the roll nip portion, which is essentially parallel to the roll axes, ensures good clamping of the material and higher compression. This creates greater material stability in the bonding portion that runs parallel to the roll axes.

For example, it can be provided that a roll nip thickness in the first and/or second conical bonding portion is between 0.02 mm and 0.08 mm, preferably 0.05 mm.

Furthermore, a height of the first conical bonding portion can be between 0.2 mm and 0.6 mm, preferably 0.4 mm. As a result, the opening in the material has a circular conical ring structure of bonded material surrounding the opening.

Furthermore, a height of the second conical bonding portion can be between 0.2 mm and 0.6 mm, preferably 0.4 mm. As a result, the embossed structure in the material has a circular conical ring structure made of bonded material that surrounds the embossed structure.

It can be provided that a width of the bonding portion formed substantially parallel to the roll axes is between 0.5 and 1 mm, preferably 0.7 mm. The material regions that are bonded parallel to the material plane and surround the openings in a brim-like manner also ensure that the three-dimensional material structure of the sheet material is more resistant. By adjusting the size of the bonding portion, which is essentially parallel to the material plane, a basic strength of the sheet material to be produced can be configured.

It is conceivable that an angle of the first conical bonding portion to the vertical is between 10° and 18°, preferably 14°.

In addition, it can be provided that an angle of the second conical bonding portion to the vertical is between 10° and 18°, preferably 14°.

Furthermore, it can be provided that the first conical bonding portion is aligned parallel to the second conical bonding portion.

Furthermore, it can be provided that a device for heating the roll surface is provided in the first and/or in the second embossing roll. By setting an optimal embossing temperature in conjunction with a predetermined embossing pressure, the desired three-dimensional sheet material structure can be produced particularly advantageously at a high process speed.

The invention further relates to a windable, in particular pre-bonded sheet material, which comprises:
at least one pre-bonded sheet material layer extending in one plane;
a plurality of structural elements distributed on the sheet material layer;
wherein the structural elements each have a border which at least in portions surrounds the structural element and which is formed by a local bonding of the sheet material, wherein the border has at least a first conical portion and at least one portion formed substantially parallel to the plane.

The border can have a stepped profile. The stepped profile may have a plurality of conical portions and a plurality of portions formed substantially parallel to the plane.

It can be provided that the first conical portion is immediately adjacent to the opening. Furthermore, the portion formed substantially parallel to the plane can directly adjoin the first conical portion.

Furthermore, it can be provided that the border has a second conical portion which, opposite the first conical portion, adjoins the portion formed substantially parallel to the plane. It can be provided that the first conical portion encloses the structural element in a ring shape. Alternatively, the portion formed substantially parallel to the plane may surround the first conical portion in portions. The structural elements can be designed as openings. The structural elements can be designed as elevations. The portion which is formed substantially parallel to the plane can be provided anywhere on the sheet material layer where it does not have any structural element such as elevations, indentations or openings.

Furthermore, it can be provided that the sheet material further has a plurality of elevations extending away from the plane.

Furthermore, it can be provided that the second conical portion also directly adjoins an elevation.

Exemplary embodiments of the invention are explained on the basis of the following figures. In the figures:

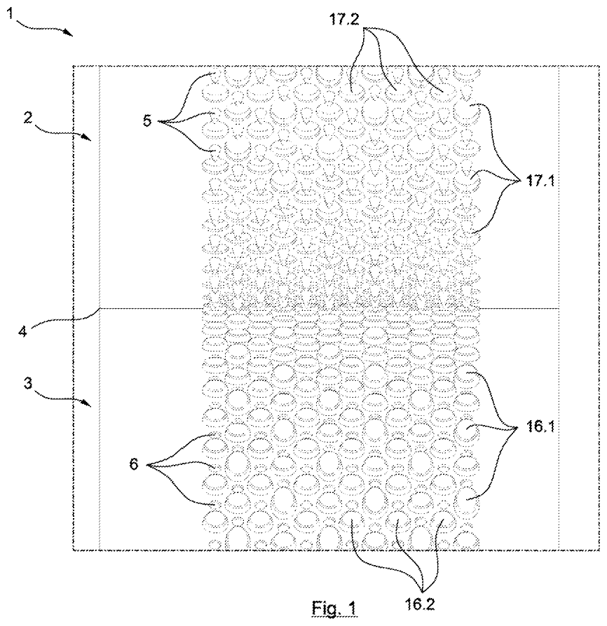

FIG. 1 shows a front view of an embodiment of the roll assembly according to the invention;

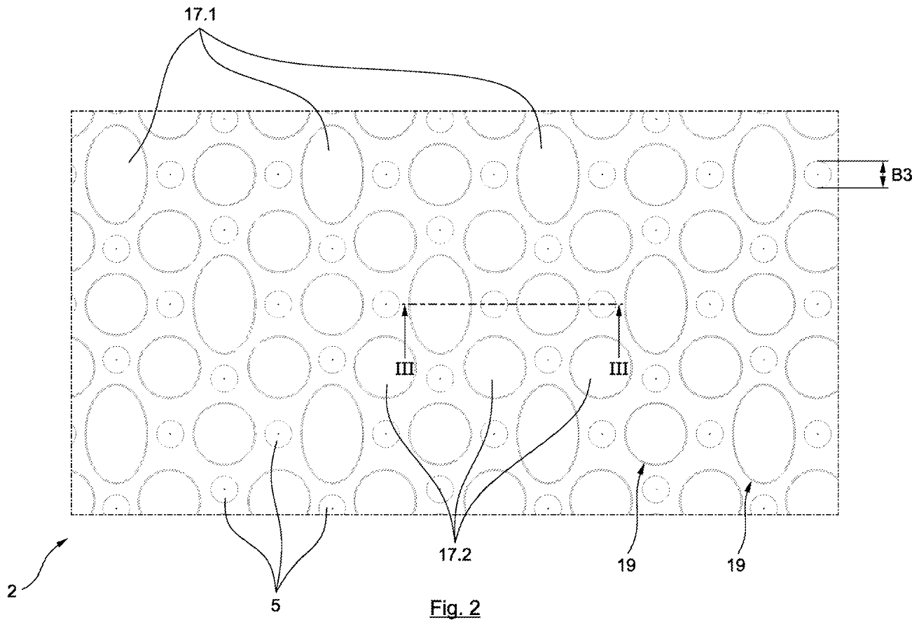

FIG. 2 shows a plan view of a roll portion of an embodiment of the first embossing roll;

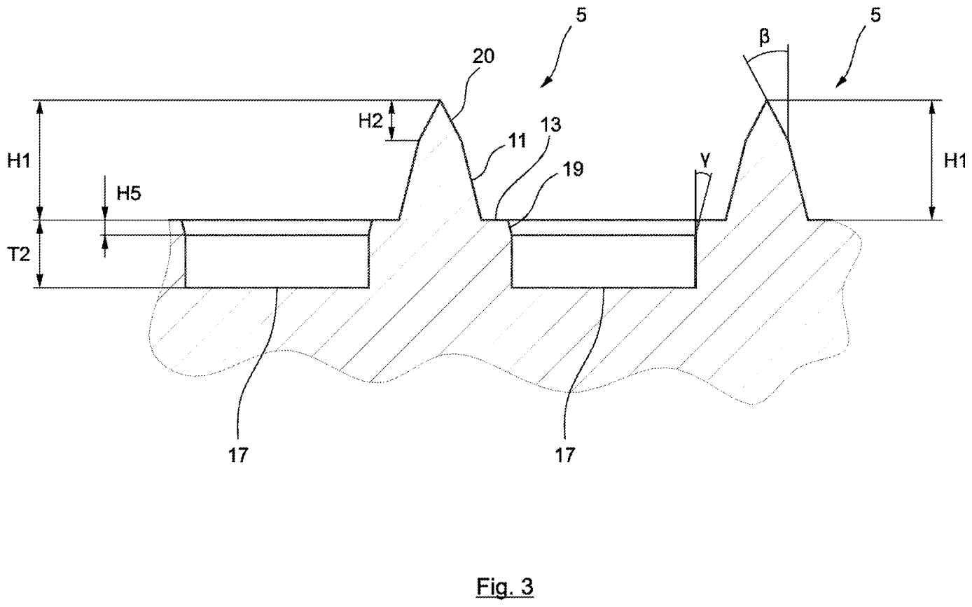

FIG. 3 shows an enlarged cross sectional view of an embodiment of the first embossing roll;

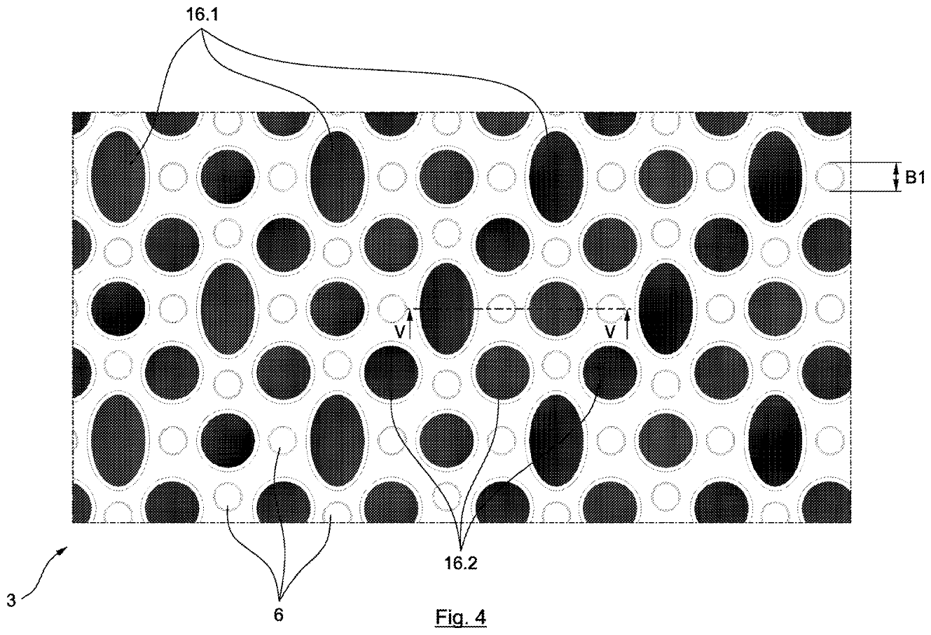

FIG. 4 a plan view of a roll portion of an embodiment of the first second embossing roll;

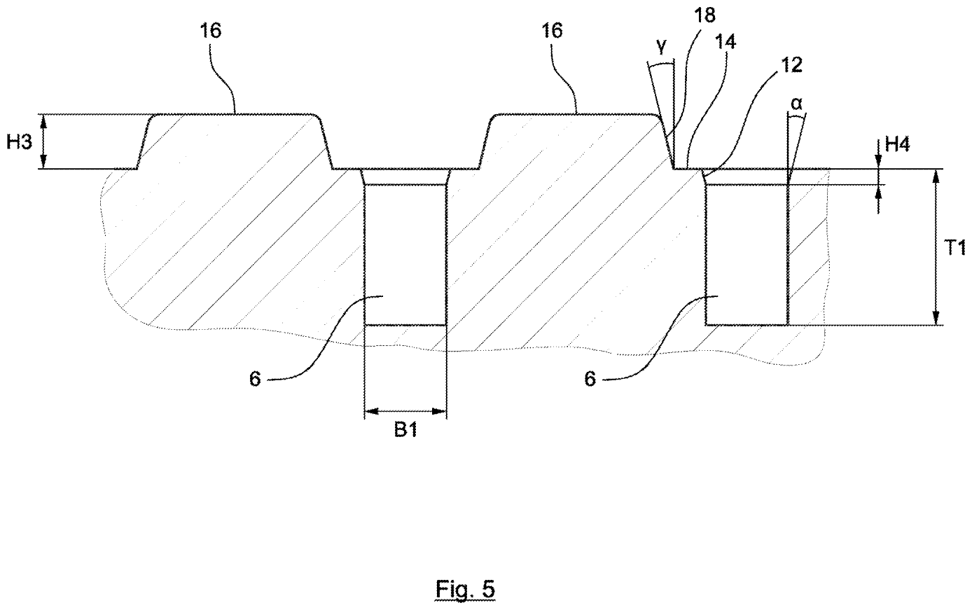

FIG. 5 shows an enlarged cross sectional view of an embodiment of the second embossing roll; and

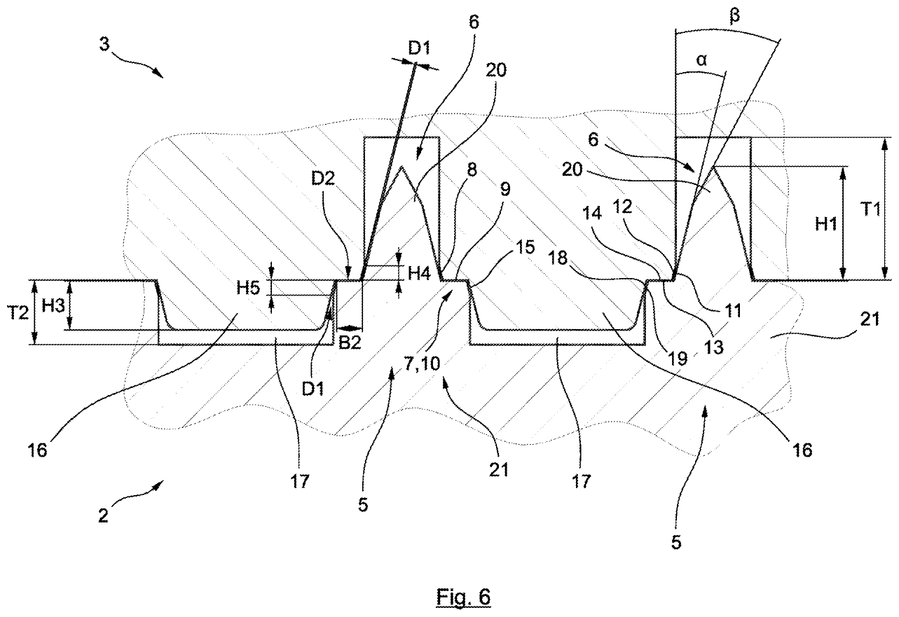

FIG. 6 shows an enlarged cross sectional view of an embodiment of the embossing nip formed between the first and second embossing roll;

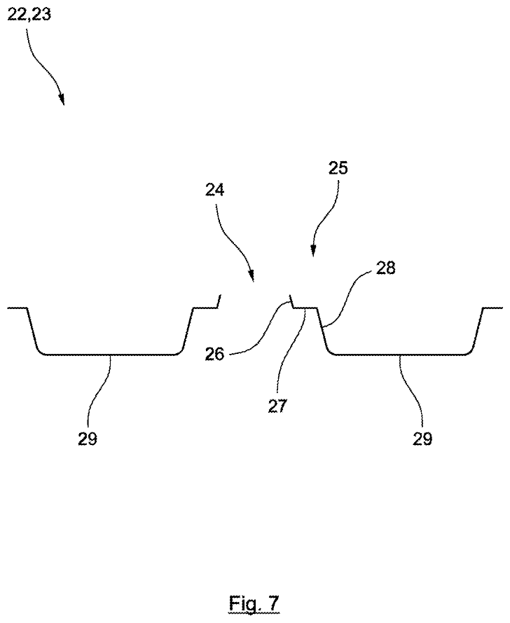

FIG. 7 shows an exemplary embodiment of a windable, in particular pre-bonded, sheet material in a cross sectional view;

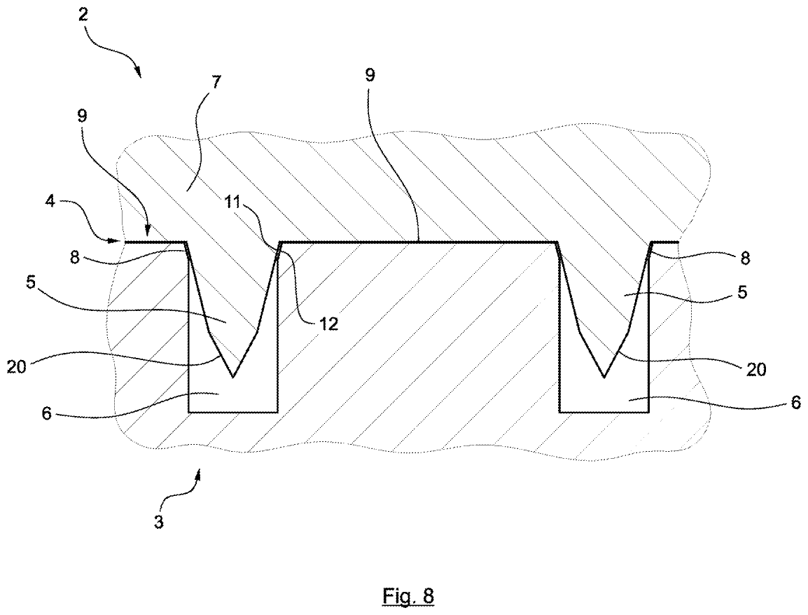

FIG. 8 shows an enlarged cross sectional view of a further embodiment of the embossing nip formed between the first and second embossing roll;

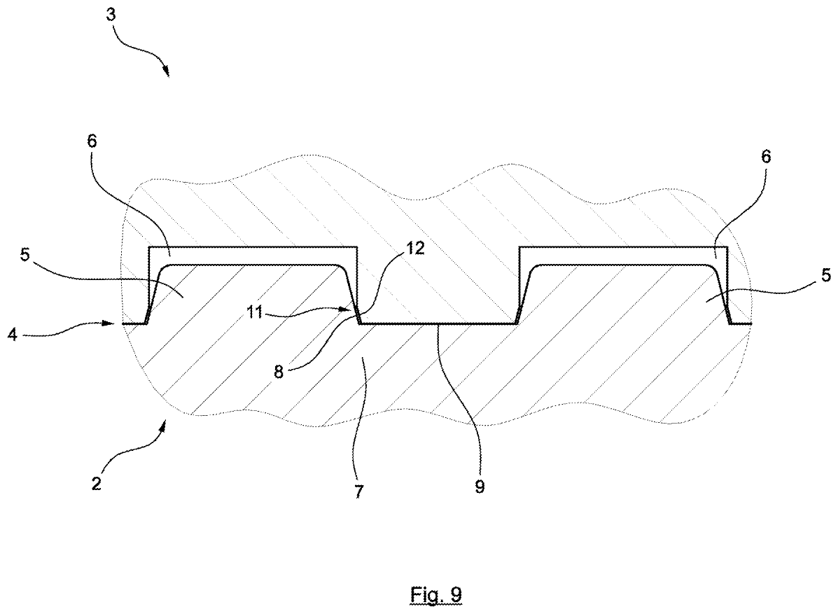

FIG. 9 shows an enlarged cross sectional view of a further embodiment of the embossing nip formed between the first and second embossing roll.

The roll assembly 1 shown in FIG. 1 has the first embossing roll 2 and the second embossing roll 3 forming a roll nip 4. It can be seen that a plurality of needles 5 are arranged in a regular distribution on the first embossing roll 2. The needles 5 are at the same distance from all neighboring needles 5. Of course, the needles 5 can also have different distances from each other. A plurality of first recesses 17.1 with an oval cross section and a plurality of second recesses 17.2 with a round cross section are arranged between the needles. The ratio of first to second recesses 17.1, 17.2 is 1:3. On the second embossing roll 3, embossing structures complementary to the structures of the first embossing roll are formed. On the one hand, the second embossing roll has a plurality of indentations 6 which are aligned such that when the rolls 2, 3 roll on one another, the needles 5 are received in the indentations 6. In an equally complementary arrangement, the second embossing roll further comprises a plurality of first embossing elevations 16.1 and a plurality of embossing elevations 16.2, which engage in the corresponding recesses 17.1, 17.2 in the roll nip 4. Corresponding to the recesses 17.1, 17.2, the first embossing elevations 16.1 have an oval cross section and the second embossing elevations 16.2 have a round cross section.

FIG. 2 shows a plan view of the embossing structure of the first embossing roll 2. The embossing structure has a plurality of needles 5, furthermore a plurality of first recesses 17.1 with an oval cross section and a plurality of second recesses 17.2 with a round cross section. It can be seen that each needle 5 is surrounded by three second recesses 17.2 with a round cross section and a first recess 17.1 with an oval cross section. The first recess 17.1 with an oval cross section can be arranged to the left, right, above or below the needles 5. The largest width B3 of the needles 5, that is, the width of the foot region of the needles 5 adjacent to the base region 21, is 2.2 mm in the embodiment shown. The first and second recesses 17.1, 17.2 each have a conical mouth portion 19 on their upper edge facing the roll surface, which mouth portion is designed in the form of a bevel surrounding the recesses in a ring shape. The width of the conical mouth portion 19 of both recesses 17.1, 17.2 is 0.2 mm in the embodiment shown.

FIG. 3 shows an enlarged cross sectional view of the embossing roll structure of the first embossing roll 2. In particular, a cross sectional view of the needles 5 can be seen. The needle 5 is arranged on a base region 21 and essentially has a conical shape extending away from the base region 21. The base region has a greater width than the width B3 of the foot region of the needles 5. The upper side of the base region 21 lies on the level of the pitch circle diameter of the rolls. The height H1 of the needles 5 is 3.2 mm in the embodiment shown. On the top of the needles 5 there is a needle tip 20 with a height H2, which in the illustrated embodiment is 1.07 mm. The needle tip 20 is less acute with a flank angle β than the lower region of needles 5, which has a flank angle α, where β>α. In the example shown, α is 14° and β is 28°. The lower region of the needles 5 provides a conical proximal flank portion 11 in which the first conical portion of the border of the opening of the sheet material is produced. The essentially horizontal saddle region 13 of the needles 5 is formed by the upper sides of the base region 21 which project horizontally beyond the needle cross section and serves to produce the essentially horizontally formed bonding portion 9 of the border. Conical mouth portions 19 of the four recesses 17.1, 17.2 surrounding the needle 5 adjoin the saddle region 13. These then form, together with the complementary embossing elevations 16.1, 16.2, the second conical bonding portion 15 in portions. The recesses 17 each have a depth T2, which in the example shown is 1.8 mm. The height H5 of the conical mouth portion 19 of the recesses 17 is 0.4 mm in the embodiment shown. The conical mouth portion has an angle γ with respect to the vertical, which in the example shown is 14°.

FIG. 4 shows a plan view of the embossing structure of the second embossing roll 3. The embossing structure has a plurality of indentations 6, furthermore a plurality of first embossing elevations 16.1 with an oval cross section and a plurality of second embossing elevations 16.2 with a round cross section. According to FIG. 2, it can be seen that each indentation 6 is surrounded by three second embossing elevations 16.2 with a round cross section and a first embossing elevation 16.1 with an oval cross section. The first embossing elevation 16.1 with an oval cross section can be arranged to the left, right, above or below the needles 5. The width B1 of the indentation 6, i.e. the width of the indentation cylinder extending into the roll surface, is 2.1 mm in the embodiment shown. The first and second embossing elevations 16.1, 16.2 each have a rounding on their upper edge pointing away from the roll surface, which in the embodiment shown has a radius of 0.3 mm. The width of the conical mouth portion 12 of the indentation 6 is 0.2 mm in the embodiment shown.

FIG. 5 shows an enlarged cross sectional view of the embossing roll structure of the second embossing roll 3. In particular, a cross sectional view of the indentations 6 can be seen. The indentations 6 are arranged between the embossing elevations 16 and essentially have a cylindrical shape extending into the roll surface with a width B1 and a depth T1, wherein in the embodiment shown the width B1 is 2.1 mm and the depth T1 is 4 mm. At the upper edge of the mouth region of the indentation 6, the latter has a width of 2.3 mm. This width is therefore greater than the largest width B3 of the needles 5 and the depth T1 is greater than the height H1 of the needles 5. The mouth angle of the mouth region 12 has an angle α of 14°. The shoulder region 14 surrounding the upper edge of the indentation lies on the plane of the pitch circle diameter of the rolls. The conical mouth region 12 of the indentation 6 serves in cooperation with the conical proximal flank portion 11 of the needles to produce the first conical portion of the border of the opening of the sheet material. Together with the horizontal saddle region 13 of the needles 5, the essentially horizontal shoulder region 14 of the indentation 6 serves to produce the essentially horizontally formed bonding portion 9 of the border. Conical flank portions 18 of the embossing elevations 16 of the four embossing elevations 16.1, 16.2 surrounding the indentation 6 adjoin the shoulder region 14. Their proximal region then form, together with the complementary recesses 17.1, 17.2, in portions the second conical bonding portion 15. The embossing elevations 16 each have a height H3, which in the example shown is 1.4 mm. The conical flank portions 18 of the embossing elevations each have an angle γ with respect to the vertical, which in the example shown is 14°.

FIG. 5 finally shows an enlarged cross sectional view of an embodiment of the embossing nip 4 formed between the first and the second embossing roll 2, 3. When the embossing rolls 2, 3 roll against each other, the embossing elevations 16 of the second embossing roll 3 engage in the recesses 17 of the first embossing roll 2 when passing through the embossing nip 4. The second conical bonding portion 15 is formed in the regions where the conical proximal flank portions 18 of the embossing elevations 16 and the conical mouth portions 19 of the recesses 17 meet, which are aligned parallel to each other. This has a height of H 5. The bonding portion 15 is delimited on the upper side by the substantially horizontal shoulder region 14 of the indentation 6 and on the lower side by the fact that the embossing nip widens due to the vertical course of the side wall of the recess 17 adjoining the bonding portion 15 below. The bonding portion 15 has an embossing nip thickness D1 of 0.05 mm.

When the embossing rolls 2, 3 roll against each other, the needles 5 of the first embossing roll 2 also engage in the indentations 6 of the second embossing roll 3 when passing through the embossing nip 4. The essentially horizontal bonding portion 9 is formed in the regions where the essentially horizontal saddle regions 13 of the base region 21 and the essentially horizontal shoulder regions 14 of the indentations 6 meet, which are aligned parallel to each other. This has a width of B2. The bonding portion 9 is delimited on the side facing away from the needle 5 by the conical proximal flank portion 18 of the embossing elevation 16 and on the side facing the needle 5 by the conical proximal flank portion 11 of the needles 5. The bonding portion 9 has an embossing nip thickness D2 of 0.02 mm and thus produces a greater clamping and a greater material compression than the conical bonding portions 15 and 8.

The first conical bonding portion 8 is formed in the regions where the conical proximal flank portions 11 of the needles 5 and the conical mouth portions 12 of the indentation 6 meet, which are aligned parallel to one another. It has a height H4. The bonding portion 8 is delimited on its underside by the essentially horizontal saddle region 13 of the base region 21 and on its upper side by the fact that the embossing nip widens due to the vertical course of the side wall of the indentation 6 adjoining the bonding portion 8 from above. The first conical bonding portion 8, like the second conical bonding portion 15, has an embossing nip thickness D2 of 0.05 mm. The adjacent bonding portions 8, 9, 15 form a stepped profile 10.

FIG. 7 shows an exemplary embodiment of a windable, in particular pre-bonded, sheet material 22 in a cross sectional view. The sheet material 22 has a windable sheet material layer 23 extending in a plane, which has a plurality of openings 24 extending through the sheet material layer 23. The openings 24 each have a border 25 surrounding the opening 24, which is characterized by a local bonding of the sheet material 22. The border 25 has a first conical portion 26 and a portion 27 which is formed substantially parallel to the plane. Furthermore, the border 25 has a second conical portion 28 which, opposite the first conical portion 26, adjoins the portion 27 which is formed substantially parallel to the plane. As a result, the border 25 has a stepped profile. The first conical portion 26 is immediately adjacent to the opening 24. The portion 27 formed substantially parallel to the plane directly adjoins the first conical portion 26. The sheet material further comprises a plurality of elevations 29 extending away from the plane, wherein the second conical portion 28 is directly adjacent to an adjacent elevation 29.

FIG. 8 shows a further embodiment of the invention. In this case, the first embossing roll 2 has exclusively needles 5 as structural elements and the second embossing roll 3 has exclusively corresponding indentations 6. When the needles 5 engage in the indentations 6, the sheet material is bonded or perforated in the roll nip 4. In the horizontal bonding portions 9 and in the first conical bonding portions 8 adjacent thereto and surrounding the needles 5 in a ring shape in the foot region of the needles 5, the material to be perforated is clamped so that the needle tips 20 can precisely produce the openings in the material. Also in this embodiment, the first conical bonding portion 8 is formed between the conical proximal flank portion 11 of the needles 5 and the conical mouth portion of the indentation 6. The bonding portion 9, which is formed essentially parallel to the roll axes, is produced in the roll nip portion formed by the rolling circle diameters of the first and second embossing rolls 2, 3, which extends over the entire roll width wherever no needles 5 or indentations 6 are arranged.

FIG. 9 shows yet another embodiment of the invention, in which the first embossing roll 2 has exclusively embossing elevations 5 as structural elements and the second embossing roll 3 has exclusively corresponding indentations 6 as structural elements. The embossing elevations 5 engage in the indentations 6 when passing through the roll nip 4, but instead of openings they only create elevations or bulges in the material to be structured. The embossing elevations 5 each have conical flanks, the conical proximal portions 11 of which in the foot region of the embossing elevations 5 together with the conical mouth portions 12 of the indentation 6 each form the first conical bonding portion 8 of the roll nip 4. The conical bonding region 8 encloses the entire embossing elevation 5 in a ring shape. The bonding portion 9, which is formed essentially parallel to the roll axes, is produced, according to the embodiment of FIG. 3, in the roll nip portion formed by the rolling circle diameters of the first and second embossing rolls 2, 3, which extends over the entire roll width wherever no needles 5 or indentations 6 are arranged.

The features of the invention disclosed in the above description, in the figures, and in the claims can be essential for the implementation of the invention both individually and in any combination.

### LIST OF REFERENCE NUMERALS

1 roll assembly
2 first embossing roll
3 second embossing roll
4 roll nip
5 projection, needle, embossing elevations
6 indentation
7 roll nip portion
8 first conical bonding portion
9 bonding portion formed essentially parallel to the roll axes
10 stepped profile
11 conical proximal flank portion of the needles
12 conical mouth portion of the indentation
13 saddle region of the base region formed essentially parallel to the roll axes
14 shoulder region of the indentation formed essentially parallel to the roll axes
15 second conical bonding portion
16 embossing elevations
16.1 embossing elevations with first cross section
16.2 embossing elevations with second cross section
17 recesses
17.1 recesses with first cross section
17.2 recesses with second cross section
18 conical proximal flank portion of the embossing elevation
19 conical mouth portion of the recess
20 needle tip
21 base region
24 opening
25 border
26 first conical portion
27 portion formed substantially parallel to the plane
28 second conical portion
29 elevation
B1 width of indentation
B2 width of the portion formed substantially in parallel with the roll axes
B3 largest width of needles
D1 conical embossing nip thickness
D2 horizontal embossing nip thickness
H1 needle height
H2 needle tip height
H3 height of embossing elevation
H4 height of conical proximal flank portion of the embossing elevation
H5 height of conical mouth portion of recess
T1 depth of indentation
T2 depth of recess
α angle of conical proximal flank portion of needles
β angle of needle tip
γ angle of conical mouth portion of indentation

1. A roll assembly for finishing a windable, in particular pre-bonded sheet material, comprising
a first and a second embossing roll which form a roll nip therebetween for embossing the sheet material;
wherein the first roll has a plurality of projections for producing structural elements in the sheet material and the second roll has a plurality of indentations for receiving the projections;
wherein a roll nip portion formed between the first and the second embossing roll is designed to produce a border of locally bonded sheet material which surrounds the structural element at least in portions, wherein the roll nip portion has at least one first conical bonding portion, characterized in that the roll nip portion further has at least one bonding portion formed substantially in parallel with the roll axes.

2. The roll assembly according to claim 1, wherein the roll nip portion has a stepped profile.

3. The roll assembly according to claim 1, wherein the first conical bonding portion is formed between a conical proximal flank portion of the projections and a conical mouth portion of the indentation.

4. The roll assembly according to claim 1, wherein the bonding portion formed substantially parallel to the roll axes is formed between a saddle region of the projections aligned substantially parallel to the roll axes and a shoulder region of the indentation aligned substantially parallel to the roll axes.

5. The roll assembly according to claim 1, wherein the first conical bonding portion directly adjoins the bonding portion formed substantially parallel to the roll axes.

6. The roll assembly according to claim 5, wherein the first conical bonding portion encloses the structural element in a ring shape.

7. The roll assembly according to claim 5, wherein the bonding portion aligned substantially parallel to the roll axes partially surrounds the first conical bonding portion in portions.

8. The roll assembly according to claim 1, wherein the projections are needles which are provided for producing structural elements designed as openings.

9. The roll assembly according to claim 1, wherein the projections are embossing elevations which are provided for producing structural elements designed as elevations.

10. The roll assembly according to claim 1, wherein the roll nip portion further comprises a second conical bonding portion which is opposite the first conical bonding portion and adjoins the bonding portion formed substantially parallel to the roll axes, wherein the first and the second conical bonding portion 15) extend in opposite directions away from the bonding portion formed substantially parallel to the roll axes.

11. The roll assembly according to claim 1, wherein the second roll has a plurality of embossing elevations for producing elevations in the sheet material and the first roll has a plurality of recesses for receiving the embossing elevations.

12. The roll assembly according to claim 1, wherein the second conical bonding portion is formed between a conical proximal flank portion of the embossing elevation and a conical mouth portion of the recess.

13. The roll assembly according to claim 1, wherein a roll nip thickness (D2) in the bonding portion formed substantially parallel to the roll axes is between 0.01 mm and 0.03 mm, preferably 0.02 mm.

14. The roll assembly according to claim 1, wherein a roll nip thickness (D1) in the first and/or in the second conical bonding portion is between 0.02 mm and 0.08 mm, preferably 0.05 mm.

15. The roll assembly according to claim 1, wherein a height (H4) of the first conical bonding portion is between 0.2 mm and 0.6 mm, preferably 0.4 mm.

16. The roll assembly according to claim 10, wherein a height (H5) of the second conical bonding portion is between 0.2 mm and 0.6 mm, preferably 0.4 mm.

17. The roll assembly according to claim 1, wherein a width (B2) of the bonding portion formed substantially parallel to the roll axes is between 0.5 mm and 1 mm, preferably 0.7 mm.

18. The roll assembly according to claim 1, wherein an angle (α) of the first conical bonding portion to the vertical is between 10° and 18°, preferably 14°.

19. The roll assembly according to claim 10, wherein an angle (γ) of the second conical bonding portion to the vertical is between 10° and 18°, preferably 14°.

20. The roll assembly according to claim 11, wherein the first conical bonding portion is aligned parallel to the second conical bonding portion.

21. The roll assembly according to claim 1, wherein a device for heating the roll surface is provided in the first and/or in the second embossing roll.

22. A windable, in particular pre-bonded, sheet material, comprising:
at least one windable sheet material layer extending in a plane;
a plurality of structural elements distributed on the at least one sheet material layer;
wherein the structural elements each have a border which, at least in portions, surrounds the structural element and which is formed by a local bonding of the sheet material, wherein the border has at least a first conical portion, characterized in that the border also has at least a portion which is formed substantially parallel to the plane.

23. The sheet material according to claim 22, wherein the border has a stepped profile.

24. The sheet material according to claim 22, wherein the first conical portion directly adjoins the structural feature.

25. The sheet material according to claim 22, wherein the portion formed substantially parallel to the plane directly adjoins the first conical portion.

26. The sheet material according to claim 25, wherein the first conical portion encloses the structural element in a ring-like manner.

27. The sheet material according to claim 25, wherein the portion formed substantially parallel to the plane surrounds the first conical portion in portions.

28. The sheet material according to claim 22, wherein the structural elements are formed as openings.

29. The sheet material according to claim 22, wherein the structural elements are formed as elevations.

30. The sheet material according to claim 22, wherein the border has a second conical portion which is opposite the first conical portion and adjoins the portion which is formed substantially parallel to the plane.

31. The sheet material according to claim 22, wherein the sheet material further comprises a plurality of elevations extending away from the plane.

32. The sheet material according to claim 30, wherein the second conical portion further directly adjoins an elevation.
